(19) United States
(12) Patent Application Publication
(10) Pub. No.: US 2019/0328353 A1
(43) Pub. Date: Oct. 31, 2019

(54) SYSTEM AND METHOD FOR PERFORMING ULTRASOUND AND PRESSURE MEASUREMENTS

(71) Applicant: KONINKLIJKE PHILIPS N.V., EINDHOVEN (NL)

(72) Inventors: Cornelis Petrus HENDRIKS, Eindhoven (NL); Achim Rudolf HILGERS, Alsdorf (DE); Daan Anton VAN DEN ENDE, Breda (NL); Mark Thomas JOHNSON, Arendonk (BE); Franciscus Johannes Gerardus HAKKENS, Eersel (NL)

(21) Appl. No.: 16/473,681

(22) PCT Filed: Jan. 2, 2018
(86) PCT No.: PCT/EP2018/050007

§ 371 Date: Jun. 26, 2019

(30) Foreign Application Priority Data
Jan. 5, 2017 (EP) 17150420.2

(51) Int. Cl. A61B 8/04; A61B 8/06; A61B 8/08; A61B 5/021

(52) U.S. Cl. CPC A61B 8/04; A61B 8/06; A61B 8/0891; A61B 8/12; A61B 5/02133; A61B 8/0883; A61B 8/488

(57) ABSTRACT

The invention relates to a system and a method for performing ultrasound and pressure measurements, wherein furthermore a related transducer unit and a related processor unit are concerned. Particularly a system is provided to measure blood pressure and flow in combination with a simple imaging modality, based on a single EAP sensor-actuator. A key aspect of embodiments in this regard is the electronics to switch between the different modalities.

### FIELD OF THE INVENTION

The present invention relates to a system and a method for performing ultrasound and pressure measurements, wherein furthermore a related transducer unit and a related processor unit are concerned.

### BACKGROUND OF THE INVENTION

An area in which a measuring of flow and pressure, and more specifically of blood flow and of blood pressure, is of interest is stenosis evaluation.

Stenosis is a form of arterial disease wherein the blood flow is restricted due to a local narrowing of the blood vessel, e.g. due to plaque formed at the vessel wall. Stenosis evaluation is known to be supported with guidewire sensors (CMUT, piezo-crystal, resistors) which either measure local blood flow, or local blood pressure. However, the complex hemodynamics of stenosis is not sufficiently explained by either pressure or flow alone. Therefore wires are being developed with multiple sensors. This, however, leads to complex devices.

After sensing stenosis, treatments like stenting are applied. It is important to accurately place a stent in the vessel (longitudinal with respect to the problem area, but also radially, i.e. the interaction with the stenosis/vessel wall (including amount of stretching)). For this purpose imaging is useful, for instance intravascular imaging.

Systems and methods for user control over the acquisition, processing, and presentation of medical data are provided in US 2014/0180087 A1. Some embodiments are particularly directed to controlling the display of multi-modality medical data in a multi-modality processing system. In one embodiment, a medical imaging system receives a set of medical data including a first data subset collected using a first sensor and a second data subset collected using a second sensor, where the first sensor and the second sensor are different. A display attribute to be applied to the first data subset independent of the second data subset is received. An instruction is generated that affects the processing of the first data subset based on the display attribute. The first data subset is processed according to the instruction. The processed first data subset is displayed according to the display attribute, and the second data subset is displayed independent of the display attribute.

In view of the above, it is desirable to have a single and simple multifunction component for sensing pressure, sensing flow and imaging.

### SUMMARY OF THE INVENTION

It is an object of the present invention to provide a simple way of performing ultrasound and pressure measurements which avoids the complexities of known systems including multiple guidewire sensors.

In a first aspect of the present invention a system for performing ultrasound and pressure measurements is presented, the system comprising a transducer unit including a sensor element made of an electro active polymer, and a processor unit coupled to the transducer unit, wherein the processor unit is arranged for processing sensor data obtained by the sensor element, wherein the system is arranged for performing ultrasound measurement and pressure measurements using the sensor element.

In a second aspect of the present invention, a transducer unit for performing ultrasound and pressure measurements is presented, the transducer unit comprising a sensor element made of an electro active polymer, wherein the sensor element includes a transmission portion and a receiving portion, wherein the receiving portion is arranged for ultrasound and pressure measurements during transmission of ultrasound from the transmission portion.

In a third aspect of the present invention, a processor unit for processing ultrasound and pressure measurement signals is presented, the processor unit comprising an interface for receiving signals from a transducer unit, an ultrasound processing element for processing ultrasound measurement signals, and a pressure processing element for processing pressure measurement signals, wherein the interface is arranged for selectively forwarding portions of received signals to either the ultrasound processing unit or the pressure processing unit.

In a fourth aspect of the present invention, a method of performing ultrasound and pressure measurements is presented, using a transducer unit including a sensor element made of an electro active polymer, wherein both, the ultrasound measurements and the pressure measurements, are performed using the sensor element.

Electro active polymers (EAP) are an emerging class of materials within the field of responsive materials. EAPs can work as sensors or actuators and can easily be manufactured into various shapes allowing easy integration into a large variety of systems.

Material characteristics such as actuation stress and strain have improved significantly over the last 10 years and technology risks have been reduced to acceptable levels for product development. Advantages of EAPs include low power requirements, small form factor, flexibility, noiselessness, accuracy, high resolution, fast response and reversible actuation. The improved performance and particular advantages of EAP material allows new and superior applications, provided that EAP modules are optimized for function performance and system integration.

Electro active polymers can be subdivided in field driven and ionic driven materials. Examples of field driven EAPs are electrostrictive polymers such as dielectric elastomers, PVDF based relaxor polymers and liquid crystal elastomers (LCE). Examples of ionic driven EAPs are conjugated polymers, carbon nanotube (CNT) polymer composites and Ionic Polymer Metal Composites (IPMC). Field driven EAP's are actuated by an electric field through direct electromechanical coupling, while the actuation mechanism for ionic EAP's involves the diffusion of ions. Both classes have multiple family members, each having its own advantages and disadvantages.

Basic actuator responses of field driven EAPs are known to the skilled person. An EAP provided between two flexible electrodes expands in the plane defined by the EAP with applied power, while the provision of a carrier layer (thus limiting flexibility) introduces a bending upon application of power.

EAPs can also be used as sensors using piezoelectric read out (contact pressure leads voltage output).

New copolymers of PVDF (e.g. P(VDF-TrFE)) developed over the last few years, enable application in flexible and 3-D shaped devices. Besides, P(VDF-TrFE) offers particular advantages over other materials including broadband width, mechanical flexibility, better acoustic impedance match to tissue, and lower cost. With these polymers it was found to measure catheter-based blood pressure and to generate and receive ultrasound, for instance for (Doppler) flow measurements (kHz range) and biomedical imaging (MHz range). Fabrication of PVDF ultrasound elements using low temperature lithography potentially enables downscaling in catheter and wire applications. A PVDF foil may be used for device tracking (“in situ”), via picking up sound waves from an external ultrasound transducer (in this way the position of the PVDF foil can be calculated and for instance indicated in the image of the external ultrasound transducer).

It was realized by the inventors that EAPs have multifunction capability and may be provided, for example, for combined pressure sensing and transmitting and receiving ultrasound. This invention provides particularly a system to measure blood pressure and flow in combination with a simple imaging modality, based on a single EAP sensor-actuator. A key aspect of embodiments in this regard is the electronics to switch between the different modalities.

In a preferred embodiment, a controller unit is coupled to the transducer unit, wherein the controller unit is arranged for operating the sensor element so to emit ultrasound.

The system or the processing unit according to the present invention may thus be arranged to control the emission of ultrasound, while it is also possible that the transducer unit of the present invention is only used for picking up ultrasound signals originally generated outside the system, e.g. by a separate ultrasound source.

In a preferred embodiment, the sensor element is mounted on a passive flexible foil and fixed on one side, wherein the controller unit is further arranged to provide an offset signal to the sensor element for deforming the combination of sensor element and passive flexible foil.

In a bending arrangement of the sensor element being single sided clamped and with the sensor element being located, for example, on the tip of the bending arrangement, it is possible to provide a scanning due to the changing orientation of the sensor element.

In a preferred embodiment, the sensor element is mounted on an active flexible foil made of an electro active polymer, wherein the controller unit is further arranged to control the active flexible foil for deforming the combination of sensor element and active flexible foil.

The active flexible foil may be used, in a way similar to the embodiment with the passive flexible foil, for a scanning motion of the sensor element, while in this case the internal stress responsible for the bending may be further increased by an opposite control of the sensor element (DC offset) and the active flexible foil. A preferable material (class) for the active flexible foil is a (P(VDF-TrFE-CTFE)) ter-polymer or a (P(VDF-TrFE-CFE)) ter-polymer.

In a preferred embodiment, the sensor element is provided in a double-side clamp configuration with a pre-curved element, wherein the controller unit is further arranged to provide an offset signal to the sensor element for moving the sensor element according to the pre-curve.

A pre-curved double-sided clamped configuration can be used for distance control to the tissue. By actuating a non-laminated sensor element with a DC voltage, the arrangement will bend in a predetermined direction according to the pre-curve, due to which the distance to the tissue can be controlled very precisely, giving additional advantages such as e.g. compensating motion artifacts, fine tuning the quality of the measurements, or for scanning purposes.

In a preferred embodiment, on the electro active polymer of the sensor element a patterned electrode arrangement is provided.

The sensor element can be a single foil made of an electro active polymer with a patterned electrode. The electrode can for instance be an evaporated, sputtered thin conductive film, patterned by using a mask. This enables imaging (electronically scanning) while maintaining low costs. It is an option to use some elements for (pressure) sensing and others for imaging or Doppler.

In a preferred embodiment, the ultrasound measurements include an ultrasound Doppler measurement and/or an ultrasound imaging measurement.

In a preferred embodiment, the electro active polymer is a polyvinylidene fluoride (PVDF) polymer or a PVDF based relaxor polymer, in particular a homo-polymer or a PVDF copolymer (e.g. P(VDF-trifluoroethylene) (P(VDF-TrFE)).

In a preferred embodiment, the sensor element is mounted on a backing, wherein between the receiving portion and the backing a cavity is provided.

It was found that the cavity allows for an amplification of the static pressure sensing without interfering with ultrasound sensitivity.

In a preferred embodiment, between the sensor element and the processor unit a switch is provided for connecting either to the ultrasound processing unit or to the pressure processing unit of the processor unit and/or between the sensor element and the processor unit an ultrasound signal line including a high-pass filter for forwarding ultrasound measurement signals to the ultrasound processing element and a pressure signal line including a low-pass filter for forwarding pressure measurement signals to the pressure processing element are provided.

By using a switch, the system can be used either for ultrasound measuring or for pressure measuring, so to have a time multiplexing in this regard. The provision of filtering allows for measuring pressure and ultrasound signals simultaneously, as the respective signals are separated from each other.

In an alternative to a just hardware based separation of received signals, it is also contemplated that the separation is provided based (in alternative or in addition) by means of a digital algorithm or software solution. In such case, no separate lines or filters would be needed. This may advantageously be combined with any digital post processing provided anyhow even in a case where separate lines, filters or the like are provided.

In a preferred embodiment, the pressure processing element includes a memory storing an absolute pressure value, wherein the pressure processing element is arranged for obtaining a differential pressure value by processing the pressure measurement signal and for updating the absolute pressure value according to the differential pressure value.

Even when it might not be possible to determine an absolute value of the pressure repeatedly, this aspect of the invention allows for maintaining an approximation of the absolute value due to observation of the pressure changes.

In a further aspect of the present invention a computer program or software product is presented for performing ultrasound and pressure measurements, the software product comprising program code means for causing a processor unit or system according to the invention carry out the steps of the method according to the invention when the software product is run on the processor unit or the system.

It shall be understood that the system of claim 1, the transducer unit of claim 9, the processor unit of claim 11, the method of claim 14, and the computer program of claim 15 have similar and/or identical preferred embodiments, in particular, as defined in the dependent claims.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows a system for performing ultrasound and pressure measurements in accordance with an embodiment of the invention,

FIG. 2 shows an overview of a system for performing ultrasound and pressure measurements in accordance with another embodiment of the invention,

FIG. 3 shows an overview of a system for performing ultrasound and pressure measurements in accordance with yet another embodiment of the invention,

FIG. 4 shows schematically a transducer unit according to an embodiment of the invention,

FIG. 5 shows schematically aspects of a transducer unit according to another embodiment of the invention,

FIG. 6 shows schematically aspects of a transducer unit according to yet another embodiment of the invention, and

FIGS. 7A and 7B show flow diagrams of methods of performing ultrasound and pressure measurements according to embodiments of the invention.

### DETAILED DESCRIPTION OF EMBODIMENTS

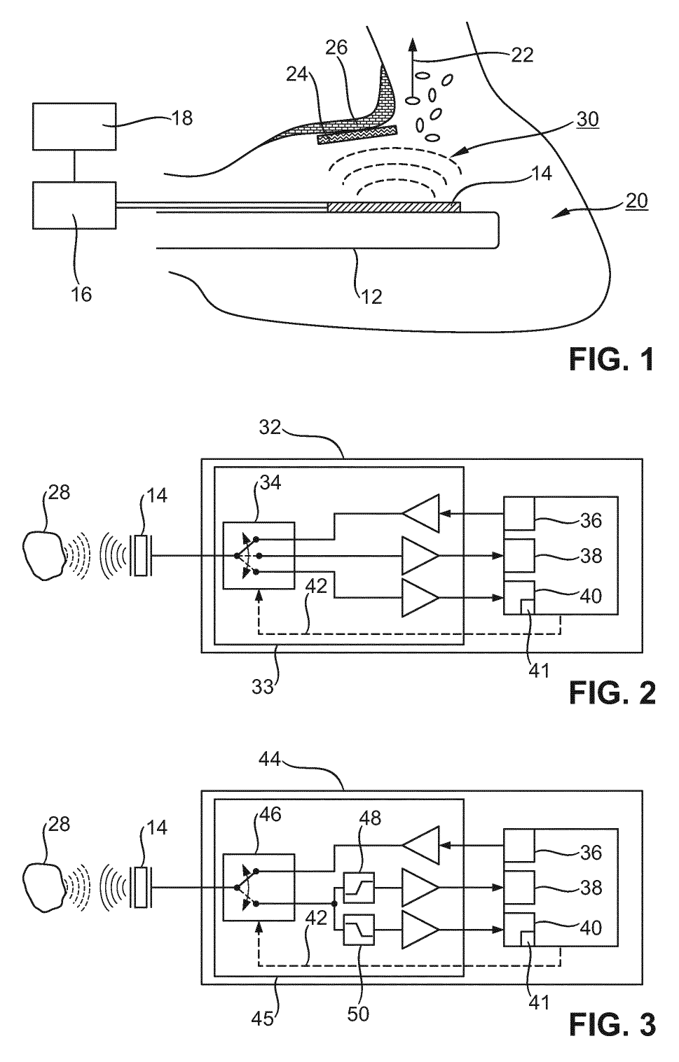

FIG. 1 shows a system for performing ultrasound and pressure measurements in accordance with an embodiment of the invention.

The system shown in FIG. 1 comprises a flexible element 12, to which a PVDF foil 14 acting as sensor element is attached. The sensor element 14 is connected to a control and drive unit 16, which is coupled to a signal processing unit 18.

The control and drive unit 16 is arranged to selectively forward received signals from the sensor element 14 to the signal processing unit 18, while details of implementations thereof are discussed below with regard to FIGS. 2 and 3.

The sensor element 14 mounted on the flexible element 12 is provided here inside a lumen or cavity 20 filled with fluid, while there is a varying pressure and/or flow. An example thereof would be a blood vessel inside a human or animal body.

The velocity of the fluid is illustrated by arrow 22.

In the lumen 20 or in its wall, there might in particular be ultrasound reflecting structure or portions 24, 26.

The ultrasound signal emitted from the sensor element 14 is illustrated by the schematic representation of waves 30.

The control and drive unit 16 is configured to sequentially apply a (pulsed) high frequency (MHz) AC signal to the PVDF foil 14 so to transmit ultrasound, to sense high frequency pressure fluctuations (reflected ultrasound echoes, or ultrasound from an external transducer), and to sense low frequency pressure variations (Hz, in particular 0 to 3 Hz, e.g. blood pressure) to measure the fluid pressure.

The signal processing unit 18 is configured to generate an ultrasound image (e.g. A-line) and/or to determine a fluid velocity from a Doppler shift and also to calculate a blood pressure (differential) based on the outputs of the sensor element 14.

The PVDF foil or sensor element 14 can be a homo-polymer or a PVDF-TrFE copolymer.

As discussed in further detail below, the configuration of the electronics of the control and drive unit may comprise a switching state to separate between the low frequency signals of the pressure measurement and the high frequency signals of the ultrasound and/or filter technology to separate between the received high frequency (ultrasound) and low frequency (pressure) signals.

FIG. 2 shows an overview of a system for performing ultrasound and pressure measurements in accordance with another embodiment of the invention.

The system includes a sensor element 14 coupled to a processor unit 32, wherein the sensor element 14 may transmit ultrasound to tissue 28 and receive ultrasound echoes.

The processor unit 32 includes a switch 34, which connects the sensor element 14 and further circuitry of the processor unit 32. In particular, the processor unit 32 includes a controller unit 36, connected to the switch 34 of an interface 33 by a line including an (optional) amplifier. Furthermore, an ultrasound processing element 38 and a pressure processing element 40 (including a memory 41 for storing an updatable absolute pressure value) are provided, which are also connected to the switch 34 by lines including (optional) amplifiers. There is further provided a control line 42 to the switch 34 for control of the setting of the switch 34.

This embodiment is based on using the three-state switch 34 (while, instead, a solution based on using two separate switches can be used as well, for example). The additional switching state (in comparison to a switch switching between ultrasound transmission and ultrasound reception) can be activated if quasi-static (qs) measurements are requested to be done (e.g. blood pressure (variation)).

The scanning or sampling rate may be different, i.e. the third switching state may be activated not as often as the receiving of the US-information but only e.g. on demand or according to a fixed (or variable) time scheme.

FIG. 3 shows an overview of a system for performing ultrasound and pressure measurements in accordance with yet another embodiment of the invention.

The embodiment shown in FIG. 3 is similar to that discussed above and shown in FIG. 2, while in this case of the processor unit 44, in an interface 45, only a two-state switch 46 is provided, wherein furthermore in the lines from the switch 46 to the ultrasound processing element 38 and to the pressure processing element 40 a high-pass filter 48 and a low-pass filter 50 are provided, respectively.

The embodiment of FIG. 3 makes use of splitting the receiving path by means of a high-pass filter (for the US-receiver) and a low-pass filter (for quasi-static (qs) measurements such as e.g. blood pressure (variation)). The filter can be realized in different ways, either based on passive or active solutions. A band pass filter can be used to further restrict the required frequency band and to improve the signal to noise behavior. The benefit of such a filter-based approach is that both receiving functions may be used at the same time (in parallel). In special cases the low-pass filter may be obsolete, if e.g. no high frequent signals can be received. Further, another option may be a software- or algorithm-based separation, i.e. providing a virtual separation of the signal rather than a hardware-implemented separation.

The discussion of the embodiment of FIG. 2 and FIG. 3 address exemplary solutions to split the receiving path into the high frequent (ultrasonic) and the low frequency (blood pressure) signals. Combinations and alternative solutions are appreciated by the person skilled in the art.

The embodiments shown in FIGS. 1 to 3 are in general useful for measuring relative pressure changes. If an absolute static pressure is needed additional measures may be taken. In particular for charge based static pressure sensing an additional measure may be taken to ensure absolute static pressure is accurately measured: the absolute pressure is recorded and stored in the electronics before switching to US transmission mode. After switching back to the sensing mode, the charge is reset to zero and the differential pressure is measured from the start of the sensing episode. The differential pressure is then added to the previously stored absolute pressure to derive an actual absolute pressure.

It is assumed that the low frequency pressure drop is small in the time that the switch is set to transmission mode, so that the accuracy of the absolute pressure measurement is maintained.

Alternatively, the low pressure measurement is resonance based, in which case the three state switch approach as described above with the illustration of FIG. 2 is preferable.

In an embodiment as shown in FIG. 1, the flexible element may be a catheter. The PVDF sensor-actuator may be used to measure local blood pressure and flow velocity. Additionally, the PVDF sensor-actuator can be used for local intravascular imaging (biomicroscopy), for instance to inspect the thickness of plaque or a blood vessel wall, or the position of a stent, or the radial expansion of the vessel after stent placement.

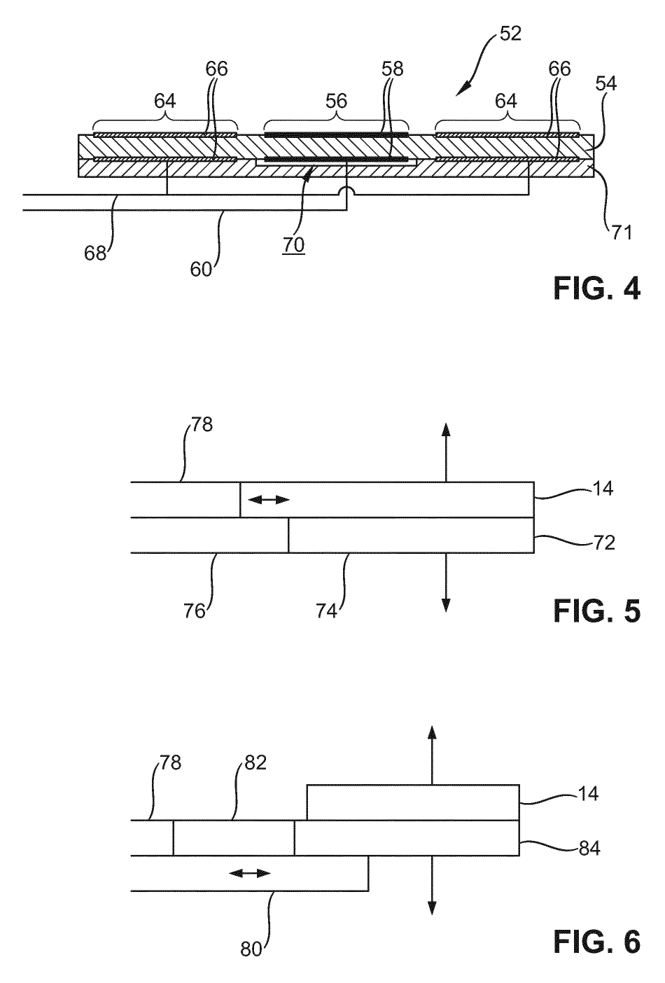

FIG. 4 shows a transducer unit 52 according to an embodiment of the invention,

The transducer unit 52 includes a single PVDF foil 54 with an area 56 for sensing (with electrodes 58) (receiving portion) and an area 64 for actuation (with electrodes 66) (transmission portion). A cavity 70 is arranged in the backing 71 under the sensing area 56 and it was found that this amplifies the static pressure sensing without interfering with the US pressure sensitivity.

The electrodes 58 for sensing are coupled to a line 60 and the electrodes 66 for actuation are coupled to a line 68.

The PVDF (co-)polymer foil 54 can be thus arranged as transmitter and sensor for parallel US transmission and sensing as well as low frequency sensing. The transmitter aspect is configured to generate US signal and the receiver aspect thereof is configured to continuously measure the static pressure and high frequency US pressure, using the filters & receiving electronics, for example as discussed above.

An advantage of electrically decoupled sensing is that the charge based sensing method can be used for continuous static measurements without the need to compute the static pressure from cumulative differential measurements when using switching electronics.

In FIG. 4, two transmission portions and one single receiving portion are shown, in contrast to, for example, the illustrations of FIG. 2 or FIG. 3. It is noted that the present invention may also be implemented just a (highly) patterned electrode arrangement, so possibly a higher number of respective transmission portions and receiving portions. In particular, it is also possible to have, within the same sheet or foil of electro active polymer more than one (separate) receiving portions, wherein one of such receiving portions may be used for receiving ultrasound signals and another one may be used for pressure measurements.

Furthermore, it is noted that generally the signal lines shown in the drawings may consist of more than only one physical signal line. Similarly, this yields also for the shown switches, instead of having only one pole (at the left/input) each physical signal line, coming from the PVDF, may end at a dedicated pole of the switch.

FIG. 5 shows schematically aspects of a transducer unit according to another embodiment of the invention.

The PVDF foil 14 forming the sensor element is laminated with a passive backing foil 72. A high voltage DC offset (ramp function) can be superimposed on the high frequency AC signal driving the ultrasound transmission in order to bend the PVDF foil 14 in cooperation with a support 78 such that the ultrasound waves can be emitted in different directions (scanning) This way 2-D images can be constructed.

In this embodiment, the passive backing foil 72 includes a flexible portion 76 and a rigid portion 74 (or alternatively a further rigid element may be added), so that the tip portion of the sensor element 14 is not also bend.

FIG. 6 shows schematically aspects of a transducer unit according to yet another embodiment of the invention.

Differing from the embodiment shown in FIG. 5, the PVDF foil 14, i.e. the sensor element, is laminated with an active backing foil 80, e.g. a (P(VDF-TrFE-CFE)) ter-polymer. Actuation of the active foil 80 induces bending (or alternatively displacement) of the PVDF foil 14 in order to provide a scanning function for 2D image construction.

Also in this embodiment, a support 78 is provided, together with a flexible portion 82 and a rigid portion 84. The expansion or compression of the active foil 80, in cooperation with the arrangement of the support 78, the flexible portion 82 and the rigid portion 84 allow for a change of direction of the sensor element 14 without a bending thereof.

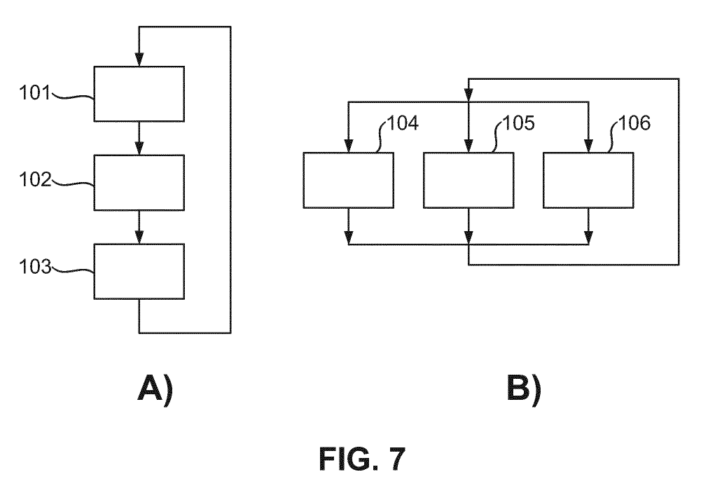

FIGS. 7A and 7B show flow diagrams of methods of performing ultrasound and pressure measurements according to embodiments of the invention.

According to the invention, a transducer element including a (single or integral) sensor element made of an electro active polymer is used for performing ultrasound and pressure measurements.

FIG. 7A shows a flow diagram of an embodiment, where, in sequence, transmission 101 of ultrasound, reception 102 of ultrasound and pressure measurement 103 are preformed.

FIG. 7B shows a flow diagram of an embodiment, where, in parallel, transmission 104 of ultrasound on the one hand and, in turn in parallel, reception 105 of ultrasound and pressure measurement 106 are preformed.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims.

For example, by using multiplexer- and/or phased delay approaches 1D-line- or even 2D-arrays can be realized.

Furthermore, a further aspect to create a low frequency stimulus from a high frequency signal may include to combine two higher frequencies with a small frequency difference (creating an amplitude envelope at the difference frequency). There might be an advantage in creating such a stimuli frequency to enhance the pick-up of the low frequency (i.e. scanning through the difference frequency from e.g. 0-10 Hz and looking for a change (e.g. Dip) in the response at the required frequency. This requires now just one (more complicated) emitter and a single receiver, but would allow distinction between high and low frequency responses.

In the claims, the word “comprising” does not exclude other elements or steps, and the indefinite article “a” or “an” does not exclude a plurality.

A single processor, device or other unit may fulfill the functions of several items recited in the claims. The mere fact that certain measures are recited in mutually different dependent claims does not indicate that a combination of these measures cannot be used to advantage.

Operations like controlling, measuring, sensing, calculating and storing can be implemented as program code means of a computer program and/or as dedicated hardware.

A computer program may be stored and/or distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

1. A system for performing ultrasound and pressure measurements, the system comprising:
a transducer unit including a sensor element made of an electro active polymer, and
a processor unit coupled to the transducer unit, wherein the processor unit is arranged for processing sensor data obtained by the sensor element,
wherein the system is arranged for performing ultrasound measurements and pressure measurements using the sensor element.

2. The system according to claim 1, further comprising:
a controller unit coupled to the transducer unit, wherein the controller unit is arranged for operating the sensor element so to emit ultrasound.

3. The system according to claim 2,
wherein the sensor element is mounted on a passive flexible foil and fixed on one side, and
wherein the controller unit is further arranged to provide an offset signal to the sensor element for deforming the combination of sensor element and passive flexible foil.

4. The system according to claim 2,
wherein the sensor element is mounted on an active flexible foil made of an electro active polymer, and
wherein the controller unit is further arranged to control the active flexible foil for deforming the combination of sensor element and active flexible foil.

5. The system according to claim 2,
wherein the sensor element is provided in a double-side clamp configuration with a pre-curved element, and
wherein the controller unit is further arranged to provide an offset signal to the sensor element for moving the sensor element according to the pre-curve.

6. The system according to claim 1, wherein on the electro active polymer of the sensor element a patterned electrode arrangement is provided.

7. The system according to claim 1, wherein the ultrasound measurements include an ultrasound Doppler measurement and/or an ultrasound imaging measurement.

8. The system according to claim 1, wherein the electro active polymer is a polyvinylidene fluoride polymer.

9. A transducer unit for performing ultrasound and pressure measurements, the transducer unit comprising a sensor element made of an electro active polymer, wherein the sensor element includes a transmission portion and a receiving portion, wherein the receiving portion is arranged for ultrasound and pressure measurements during transmission of ultrasound from the transmission portion.

10. The transducer unit according to claim 9, wherein the sensor element is mounted on a backing, wherein between the receiving portion and the backing a cavity is provided.

11. A processor unit for processing ultrasound and pressure measurement signals, the processor unit comprising:
an interface for receiving signals from a transducer unit,
an ultrasound processing element for processing ultrasound measurement signals, and
a pressure processing element for processing pressure measurement signals, wherein the interface is arranged for selectively forwarding portions of received signals to either the ultrasound processing unit or the pressure processing unit.

12. The processor unit according to claim 11, wherein
the interface includes a switch for connecting either to the ultrasound processing unit or to the pressure processing unit and/or
the interface includes an ultrasound signal line including a high-pass filter for forwarding ultrasound measurement signals to the ultrasound processing element and a pressure signal line including a low-pass filter for forwarding pressure measurement signals to the pressure processing element.

13. The processor unit according to claim 11,
wherein the pressure processing element includes a memory storing an absolute pressure value, and
wherein the pressure processing element is arranged for obtaining a differential pressure value by processing the pressure measurement signal and for updating the absolute pressure value according to the differential pressure value.

14. A method of performing ultrasound and pressure measurements, using a transducer unit including a sensor element made of an electro active polymer, wherein both, the ultrasound measurements and the pressure measurements, are performed using the sensor element.

15. A software product for performing ultrasound and pressure measurements, the software product comprising program code means for causing a processor unit to carry out the steps of the method as claimed in claim 14 when the software product is run on the processor unit.
